SEQUENCE LISTING (2 sequences)

SEQ ID NO: 1 - protein, 21 aa, Artificial Sequence
  Synthezised peptide
NDLDVPIPGRFDRRVSVAAET

SEQ ID NO: 2 - protein, 16 aa, Artificial Sequence
  Synthezised peptide
ASPQTSPWQSPAVSPK

(19) United States
(12) Patent Application Publication
(10) Pub. No.: US 2013/0059314 A1
(43) Pub. Date: Mar. 7, 2013

(54) FRET-BASED METHOD FOR THE DETERMINATION OF PROTEIN PHOSPHATASE AND KINASE ACTIVITY

(71) Applicants: Jennifer L. Gooch, Lilburn, GA (US); Brian R. Roberts, Sandy Spring, GA (US)

(72) Inventors: Jennifer L. Gooch, Lilburn, GA (US); Brian R. Roberts, Sandy Spring, GA (US)

(73) Assignee: EMORY UNIVERSITY, Atlanta, GA (US)

(21) Appl. No.: 13/517,416

(22) PCT Filed: Jan. 7, 2011
(86) PCT No.: PCT/US11/20487

§ 371 Date: Sep. 13, 2012

Related U.S. Application Data
(60) Provisional application No. 61/293,247, filed on Jan. 8, 2010.

(51) Int. Cl. G01N 21/64; C12M 1/42; C12M 1/34

(52) U.S. Cl. 435/7.4; 435/288.7

(57) ABSTRACT

This disclosure relates to methods of determining activities of protein phosphatases and kinases. The disclosure further relates to methods of clinical monitoring of calcineurin activity and immunosuppression in patients and which may be used to predict transplant acceptance in patients.

### STATEMENT ON FUNDING PROVIDED BY THE U.S. GOVERNMENT

This invention was made with government support under NIH Grant No. R01 DK066422 awarded by the U.S. National Institutes of Health of the United States government. The government has certain rights in the invention.

### FIELD OF THE DISCLOSURE

This disclosure relates to methods of determining activities of protein phosphatases and kinases. The disclosure further relates to methods of clinical monitoring of calcineurin activity and immunosuppression in patients and which may be used to predict transplant acceptance in patients.

### BACKGROUND

Calcineurin is a calcium-dependent, serine/threonine phosphatase that is a signal transduction mediator involved in a variety of pathways including T cells. There are three isoforms of the catalytic subunit of calcineurin—α, β, and γ. Unique and distinct roles for the α and β isoforms have been identified. Importantly, the β isoform appears to be the primary isoform required for normal activity of T cells.

The addition of the calcineurin inhibitors cyclosporin A and FK506 to immunosuppressive regimens reduces the incidence of acute allograft rejection and effectively doubles one-year survival of kidney transplant patients. However, long-term graft survival has improved far less significantly, with only 66% and 78% of deceased donor and living donor recipients, respectively, surviving 5 years. This statistic is even more striking when considered for different racial groups. 80% of Caucasians who receive living donor organs survive for 5 years while only 64% of African Americans live that long. Similar trends are observed for recipients of deceased donor organs; there is a 70% survival rate for Caucasians and only 55% for African Americans. Understanding mechanisms that contribute to disparate outcomes for transplant patients is an area of tremendous importance. Despite considerable effort, no consensus on the underlying causes has been reached that adequately explains racial disparities in long-term outcomes.

Cyclosporin A (CsA) and FK506 (tacrolimus) exert their immunosuppressive action by inhibition of the calcineurin. Calcineurin is known to be activated downstream of the T cell receptor and regulates transcription factors including the Nuclear Factor of Activated T cells (NFATs). NFATc proteins, in turn, control expression of cytokines including IL-2 and IL-4. Blockade of calcineurin/NFAT activity inhibits T cell activity and results in immune suppression. Although cyclosporine A has been clinically used for more than 20 years and FK506 over a decade, target blood levels for immunosuppression maintenance have yet to be properly defined. Therapeutic monitoring of trough cyclosporin concentration has proven to be a poor clinical indicator as some patients experience rejection in the presence of adequate or even high blood cyclosporin concentrations, whereas others develop toxicity even when blood trough concentrations are low. Discrepancies between cyclosporin dose and clinical immune suppression suggest that calcineurin activity itself may be a source of variability. However, there have been only limited studies that directly measure calcineurin activity, and there is no data regarding factors which may affect the calcineurin sensitivity to inhibition by cyclosporin and FK506.

### SUMMARY

This disclosure encompasses in-solution methods of detecting and measuring modifications of a peptide and methods of detecting and measuring the levels of activity of enzymes that mediate such modification reactions (e.g., phosphatases and kinases). Assays to detect such as phosphatase activity have utilized titanium oxide-coated plates to separate phosphorylated from non-phosphorylated peptide substrate. The titanium oxide bound phosphorylated peptide, or unbound dephosphorylated peptide, was then detected by a fluorescent tag that had been attached to the peptide.

While a significant advance over other methods that labeled peptides radioactively, the assay had several disadvantages, including the requirement for moving the samples from a reaction plate to a separation plate to a detection plate. In the methods of the present disclosure, the entire procedure may be completed in a single reaction. Micro-beads of titanium oxide that have been conjugated to a fluorophore such as fluorescein are used, and only the modified peptide bound to the micro-beads is quantitatively detected using Fluorescence Resonance Energy Transfer (FRET) technology. The systems of the disclosure detect only the modified peptide bound to the titanium oxide and the micro-beads may remain in suspension or be bound to a solid surface as desired.

One aspect of the present disclosure, therefore, encompasses methods for determining a peptide-modifying enzyme activity, the method comprising: (a) providing an assay reaction mix comprising a target peptide comprising an amino acid sequence specifically recognized by a peptide-modifying enzyme and a first fluorophore species conjugated to said target peptide, a buffer mix configured to allow a peptide-modifying enzyme to modify the target peptide, and a test sample suspected of comprising a peptide-modifying enzyme; (b) incubating the assay reaction mix under conditions suitable for a peptide-modifying enzyme to modify the target peptide; (c) contacting the incubated reaction mix with titanium oxide, said titanium oxide having a second fluorophore species conjugated thereon, under conditions suitable for the titanium oxide to bind to a modified target peptide but not to an unmodified target peptide; (d) illuminating the titanium oxide at an excitation wavelength of the second fluorophore species, whereby the second fluorophore species emits a first fluorescence, said first fluorescence exciting the first fluorophore species by FRET, thereby inducing the emission of a second fluorescence from the first fluorophore species, said second fluorescence having a wavelength different from the wavelength of the first fluorescence; (e) selectively detecting the second fluorescence, thereby detecting binding of a modified target peptide to titanium oxide; (f) determining the intensity of the second fluorescence; and (g) correlating the intensity of the second fluorescence to the amount of modified target peptide bound to the titanium oxide.

Another aspect of the present disclosure encompasses kits for determining the level of a peptide-modifying enzyme activity in a test sample, comprising: a container enclosing a target peptide, the target peptide comprising an amino acid sequence specifically recognized by a peptide-modifying enzyme and a first fluorophore species conjugated to the target peptide; titanium oxide conjugated to a second fluorophore species; and instructions for the use of the target peptide and the titanium oxide in determining the peptide-modifying enzyme activity of a test sample by FRET-based fluorescence measurements.

Yet another aspect of the present disclosure encompasses fluorimetric unit configured to measure a peptide-modifying enzyme activity according to a FRET-based method, comprising: a system configured to receive a plurality of assay reaction mixes, each reaction mix comprising: a target peptide comprising an amino acid sequence specifically recognized by a peptide-modifying enzyme and a first fluorophore species conjugated to said target peptide, a buffer mix configured to allow a peptide-modifying enzyme to modify the target peptide, wherein each assay mix of said plurality of assay mixes is in contact with titanium oxide having a second fluorophore species conjugated thereto, and wherein the titanium oxide is formulated as titanium oxide micro-beads or substratum-attached titanium oxide, a detection system for detecting binding of a modified target peptide to titanium oxide by FRET, wherein the titanium oxide is illuminated at an excitation wavelength of the second fluorophore species, whereby the second fluorophore species emits a first fluorescence, said first fluorescence exciting the first fluorophore species, thereby inducing the emission of a second fluorescence from the first fluorophore species, said second fluorescence having a wavelength different from the wavelength of the first fluorescence, a measuring system configured to determine the intensity of the second fluorescence, a microprocessor unit configured to correlate the intensity of the second fluorescence to the amount of modified target peptide bound to the titanium oxide; and an output unit configured to provide a measurement of the peptide-modifying enzyme activity.

### BRIEF DESCRIPTION OF THE FIGURES

Many aspects of the disclosure can be better understood with reference to the following figures. See the text and examples for a more detailed description of the figures.

FIG. 1 schematically illustrates a FRET-based assay of calcineurin activity.

FIG. 2 schematically illustrates the FRET formation of a detectable fluorescence due to binding of a phosphorylated peptide on titanium oxide micro-beads.

FIG. 3 is a graph illustrating the FRET between FLUOR-titanium oxide and TAMRA-peptide RII. FLUOR-beads alone emit a peak at approximately 520 nm; TAMRA-peptide alone emits a detectable background peak at 580 nm and the combination of the two produces a shift of the 580 nm peak.

FIGS. 4A and 4B show a pair of graphs illustrating the response of the FRET 580 nm peak as a function of FLUOR-bead concentration (FIG. 4A) or TAMRA-RII peptide concentration (FIG. 4B). In FIG. 4A, the amount of TAMRA-peptide was held constant and the amount of FLUOR-beads was increased. There was a dose-responsive increase in the FRET peak at 580 nm, and the FLUOR peak appears at 520 nm with the maximum amount of beads tested. In FIG. 4B, the amount of FLUOR-beads was held constant and increasing amounts of TAMRA-peptide RII were added. The FRET peak at 580 nm increases in a dose-responsive fashion whereas there was no change in the FLUOR peak at 520 nm.

FIG. 5A is a graph illustrating the FRET response to increasing amounts of calcineurin. The 580 nm peak was compared with the reactions containing only TAMRA-peptide RII and no beads (designated max or 100% dephosphorylation) and with a reaction containing TAMRA-peptide RII and beads, but no calcineurin (designated minimum or 0% dephosphorylation).

FIG. 5B is a graph illustrating the linear dose effect of calcineurin concentration. The curve could be used to extrapolate calcineurin activity from a range of 0 units to about 2 units under the experimental conditions.

FIG. 6 is a graph illustrating the FRET interaction between peptide RII and titanium oxide in PP1A reaction buffer. Two amounts of FLUOR-titanium oxide micro-beads were used and show a dose-effect of FRET with increasing amount of titanium oxide/RII interaction.

FIG. 7 is a graph showing that changes to the assay buffer can eliminate detection of calcineurin.

FIG. 8 is a graph showing that in the PP1A/2A buffer, a linear range of phosphatase activity not attributable to calcineurin activity was detected.

FIG. 9 is a graph showing that phosphatase activity detected in the PP1/2A buffer is sensitive to inhibition with okadaic acid, in a dose-dependent fashion. Okadaic acid is a known inhibitor if PP1/2A. Calcineurin activity is not sensitive to further reduction with a similar range of okadaic acid.

FIG. 10 is a graph illustrating the FRET response to increasing amounts of PP1A.

FIG. 11 is a graph showing that in the PP1A/2A buffer, a linear range of phosphatase activity not attributable to calcineurin activity was detected.

FIG. 12 is a graph illustrating that cyclosporine A inhibited enzyme activity in a dose-dependent manner. Jurkat cells were incubated with increasing concentrations of cyclosporine A for 30 minutes prior to harvest and calcineurin assay.

FIG. 13 is a graph illustrating that calcineurin activity increased in both a time and dose-dependent manner. Calcineurin activity was determined using 0, 0.5, or 1 unit of purified enzyme at reaction times up to 10 mins.

### DESCRIPTION OF THE DISCLOSURE

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those skilled in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Optional” or “optionally” means that the subsequently described circumstance may or may not occur, so that the description includes instances where the circumstance occurs and instances where it does not.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gin, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

By “detectably labeled” is meant that a fragment or an oligonucleotide contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, colorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, colorimetric, hybridization protection assay, and mass spectrometry

“Peptide” refers to a polymer in which the monomers are amino acid residues which are joined together through amide bonds, alternatively referred to as a polypeptide. A “single polypeptide” is a continuous peptide that constitutes the protein. When the amino acids are alpha-amino acids, either the L-optical isomer or the D-optical isomer can be used, the L-isomers being preferred. Additionally, unnatural amino acids such as beta-alanine, phenylglycine, and homo-arginine are meant to be included. Commonly encountered amino acids which are not gene-encoded can also be used in the present disclosure, although preferred amino acids are those that are encodable. For a general review, see, for example, Spatola, A. F., in Chemistry and Biochemistry of Amino Acids, Peptides and Proteins, B. Weinstein, ed., Marcel Dekker, N.Y., p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, □-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. “Amino acids” may be referred to herein by either their commonly known three letter symbols or by the one-letter symbols recommended by the IUPAC-IUB Biochemical Nomenclature Commission. Nucleotides, likewise, may be referred to by their commonly accepted single-letter codes as indicated as follows: Alanine (Ala, A), Arginine (Arg, R), Asparagine (Asn, N), Aspartic Acid (Asp, D), Cysteine (Cys, C), Glutamine (Gin, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference peptide, polypeptide, or polynucleotide, but retains essential properties. A typical variant of a peptide or polypeptide differs in amino acid sequence from another, reference peptide or polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the amino acid sequence of the peptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gin, His), (Asp: Glu, Cys, Ser), (Gln: Asn), (Glu: Asp), (Gly: Ala), (His: Asn, Gin), (lie: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

The term “peptide-modifying enzyme” as used herein refers to an enzyme that catalyzes the addition or removal of a small-molecule moiety from an amino acid of a protein or peptide. Such an enzyme may be, but not necessarily, capable of recognizing a specific amino acid sequence, thereby allowing the enzyme to attach or delete the moiety from a defined site or sites within the protein or peptide. For example, a protein kinase may add a phosphate group to a protein or peptide and a phosphatase may remove a phosphate group. Calcineurin is known as a calcium ion- and calmodulin-dependent serine-threonine phosphatase and is an element of many intracellular signaling pathways. (Guerini & Klee, (1989) Proc. Natl. Acad. Sci. USA 86:9183-9187). The protein has been identified in eukaryotic cells ranging from yeast to mammals.

The term “kinase” as used herein refers to any enzyme capable of adding a phosphate group to an amino acid side-chain of a protein, polypeptide, or a peptide.

The term “phosphatase” as used herein refers to an enzyme capable of removing a phosphate group from a protein, polypeptide, or a peptide by a hydrolytic reaction.

The term “calcineurin inhibitor” as used herein refers to a compound that in contact with calcineurin either directly or indirectly, reduces or blocks a calcineurin activity, such as, but not limited to, cyclosporine A, tacrolimus, or derivatives thereof.

The term “target peptide” as used herein refers to a peptide that can function as a substrate for a peptide-modifying enzyme. Such a peptide may comprise an amino acid sequence that is specifically recognized by the enzyme for binding to the peptide and also a site that may receive or have bound thereto a modifying moiety.

The term “to modify the target peptide” as used herein refers to the act of an enzyme adding to, or removing, from a target peptide a small-molecule moiety such as, but not limited to, an acidic moiety.

The term “FRET” as used herein refers to fluorescence resonance energy transfer between molecules. In FRET methods, one fluorophore is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g., on the same, or a neighboring molecule). FRET techniques can be readily used to detect the titanium oxide-bound peptides of the present disclosure. See for example U.S. Pat. Nos. 5,668,648, 5,707,804, 5,728,528, 5,853,992, and 5,869,255 (for a description of FRET dyes), T Mergny et al., (1994) Nucleic Acid Res. 22:920-928, and Wolf et al., (1988) Proc. Natl. Acad. Sci. USA 85:8790-8794 (for general descriptions and methods for FRET), each of which is hereby incorporated by reference in its entirety.

The term “fluorophore” as used herein refers to a fluorescent moiety that in the context of the methods of the disclosure is conjugated to a target peptide or to titanium oxide.

The term “test sample” as used herein refers to any liquid volume added to the reaction mix of the methods of the disclosure, wherein the added liquid volume comprises a known amount of a peptide-modifying enzyme, or may have (suspected) of comprising a peptide-modifying enzyme. A test sample may comprise a known amount of a peptide-modifying enzyme in a buffer suitable for allowing the enzyme to react with a target peptide, or may be derived from a biological sample such as a tissue, cell or fluid sample isolated from a human or animal subject. For example, the test sample can be, but is not limited to, a lysate prepared from isolated cells such as peripheral mononuclear blood cells, a tissue biopsy sample and the like.

The term “Peripheral Mononuclear Blood Cell(s)” as used herein refers to a blood cell having a round nucleus. For example: a lymphocyte, a monocyte or a macrophage. These blood cells are a critical component in the immune system to fight infection and adapt to intruders. The lymphocyte population consists of T cells (CD4 and CD8 positive about 75%), B cells and NK cells (about 25% combined). These cells may be extracted from whole blood using ficoll, a hydrophilic polysaccharide that separates layers of blood, with monocytes and lymphocytes forming a buffy coat under a layer of plasma. This buffy coat contains the PBMCs. PBMC can be extracted from whole blood using a hypotonic lysis which will preferentially lyse red blood cells. This method results in neutrophils and other polymorphonuclear (PMN) cells that are important in innate immune defense to be obtained.

The term “lysate” as used herein refers to a suspension of isolated cells that have had their cell membranes disrupted chemically, physically, enzymatically, or by a combination thereof. The cells may be lysed in a buffer, the disruption in the cell membranes releasing to the surrounding buffer a mix of proteins and other cell constituents. The lysis may be total, where all cells in the treated cell population procedure release their intracellular contents, or partial where at least 50%, advantageously, at least 75%, more advantageously at least 90%, and most advantageously 100% of the cells in a population of isolated cells are disrupted and release their intracellular contents into a suspension buffer.

The term “fluorescently labeled” as used herein refers to conjugating to a peptide substrate a fluorescent moiety, i.e. a fluorophore. A variety of different label moieties are available for use in the substrates of the present disclosure. Such groups include fluorescein labels, rhodamine labels, cyanine labels (i.e., Cy3, Cy5, and the like, generally available from the Amersham Biosciences division of GE Healthcare), the Alexa family of fluorescent dyes and other fluorescent and fluorogenic dyes available from Molecular Probes/Invitrogen, Inc., and described in ‘The Handbook—A Guide to Fluorescent Probes and Labeling Technologies, Tenth Edition’ (2005) (available from Invitrogen, Inc./Molecular Probes). A variety of other fluorescent and fluorogenic labels for use with nucleoside polyphosphates, and which would be applicable to the compounds of the present invention are described in, e.g., Published U.S. Patent Application No. 2003/0124576, the full disclosure of which is incorporated herein in its entirety for all purposes.

The term “selectively detecting” as used herein refers to detecting a wavelength of light from a spectrum of wavelengths. Such selection may be by, for example, filters designed to transmit light of a narrow range of wavelengths while reflecting wavelengths beyond the selected range.

### Abbreviations

TAMRA, (tetramethyl-6-carboxyrhodamine); CsA, cyclosporine(e) A; FK506, tacrolimus (FUJIMYCIN™); FRET, Fluorescence Resonant Energy Transfer.

### Discussion

Calcineurin is a calcium-dependent, serine/threonine phosphatase that is involved in a variety of signaling pathways. Calcineurin is distinct among phosphatases because its activity requires calcium and is not sensitive to inhibition by compounds that block the related phosphatases PP1A and PP2A. Therefore, the most common methods to measure calcineurin activity rely on calcium-dependent dephosphorylation of a substrate derived from the RII subunit of protein kinase A in the presence of PP1A/PP2A inhibitors.

In an established assay method for calcineurin activity, a peptide substrate is incubated with protein kinase A and ³²Py[ATP] under appropriate conditions to phosphorylate the peptide with a radioactive residue. The labeled substrate is then purified and used within a short period of time as a substrate for calcineurin. To measure calcineurin activity, equal parts of cell lysate, reaction mixture, and labeled substrate are incubated at about 30° C. for about 10 minutes before the reaction is terminated. To determine how much of the phosphorylated peptide has been dephosphorylated, for each reaction an individual column is prepared containing pre-charged ion-exchange resin. A reaction mix is loaded on the column and unincorporated phosphate, which does not bind the resin, is eluted. The amount of radioactivity in the eluted fraction is then measured in a scintillation counter and used to quantify calcineurin activity. In practice, the method has several drawbacks including the use of radioactive phosphate for labeling of the peptide substrate, background due to unincorporated phosphate, reliance upon ion exchange to separate phosphorylated from non-phosphorylated peptide, and the final measurement of free phosphate to represent calcineurin activity. These factors increase variability of the data and reduce the reproducibility of the assay.

The present disclosure encompasses non-radioactive in-solution methods for detecting and measuring modifications of a peptide and methods of detecting and measuring the levels of activity of enzymes that mediate such modification reactions (such as, but not limited to, phosphatases and kinases). Before the methods herein disclosed, an assay to detect peptide-modifying reactions such as phosphatase activity used titanium oxide-coated plates to partition phosphorylated from non-phosphorylated target peptide substrates, as disclosed in PCT Patent Application Publication No.: WO/2009/010424, incorporated herein by reference in its entirety. The titanium oxide bound phosphorylated peptide or unbound dephosphorylated peptide was then detected using a fluorophore attached to the target peptide.

Although an advance over methods that labeled peptides radioactively, this type of assay has several disadvantages, especially for high-throughput applications, due to the requirement for moving the samples from a reaction plate to a separation plate, and then to a detection plate. In the methods of the present disclosure, however, the entire procedure may be completed in a single reaction volume with no physical transfer between reaction steps. In embodiments of the disclosure, therefore, in place of a coating containing titanium oxide resin, preferably micro-beads of titanium oxide that have been conjugated to a fluorophore such as fluorescein are used, and only modified peptide that is bound to the micro-beads is quantitatively detected using Fluorescence Resonance Energy Transfer (FRET) technology.

The methods of the present disclosure may use, but are not limited to, titanium oxide in the form of micro-beads that may remain suspended in a reaction assay mix. It is contemplated, however, that the FRET-based assay of the present system may incorporate the use of titanium oxide that is attached to a substratum surface such as arrays of attached titanium oxide areas for multiple sample assaying, instead of the assay reactions being conducted in micro-well plates.

The titanium oxide micro-beads for use in the assay methods of the disclosure will have a fluorophore (the second fluorophore) conjugated to the surface of the beads. Accordingly, when a modified peptide, e.g., a phosphorylated peptide, binds to the titanium oxide micro-beads, a first fluorophore attached to the peptide is positioned adjacent to the second fluorophore that is conjugated directly on the titanium oxide. The juxtaposition of the two fluorophores, upon illuminating one fluorophore at its excitation wavelength, results in FRET between the two fluorophores, and the emission of a detectable and distinct wavelength of second fluorescence light. It is further contemplated that in embodiments of the assay systems of the disclosure, the first and second fluorophores may be selected such that the second fluorescence light may be emitted from a second fluorophore attached to the titanium oxide after excitation from the fluorescence emitted by the first fluorophore attached to the target peptide.

Embodiments of the assay systems of the disclosure may further comprise the step of relating the emitted fluorescence intensity to an (i) an enzyme activity that adds a modifying group to the target peptide, thereby increasing the amount of the peptide bonded to the micro-beads, or (ii) to an enzyme activity that removes a modifying group from a substrate peptide and thereby lessening the amount of the peptide bonded to the titanium oxide. For example, and an illustration of an application of the methods of the disclosure, a phosphorylated target peptide substrate may be provided that has a TAMRA (tetramethyl-6-carboxyrhodamine) fluorophore attached thereto. The phosphorylated target peptide can bind to the titanium oxide micro-beads via the acidic phosphorylation group under acidic conditions, whereas a dephosphorylated peptide will not. In this example, the titanium oxide can be conjugated to a second fluorophore such as, but not limited to, fluorescein.

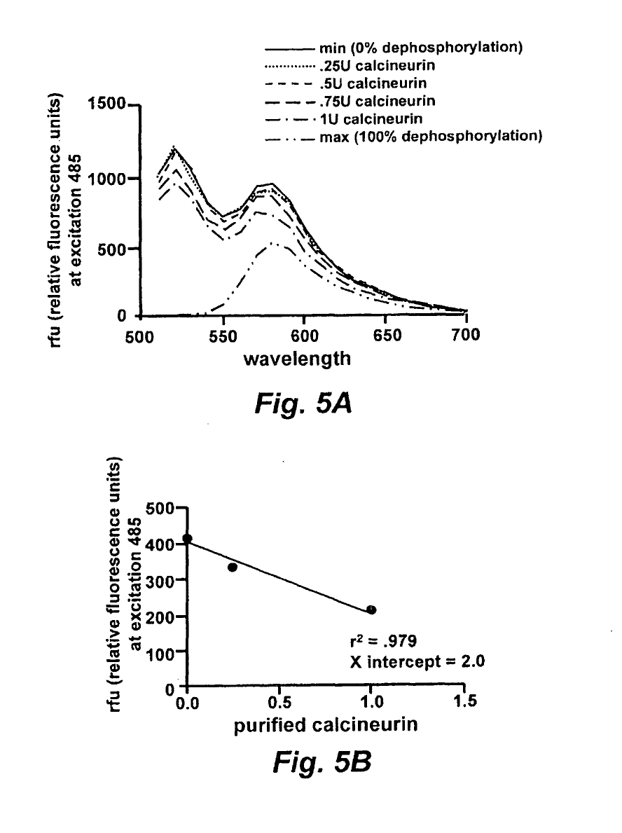

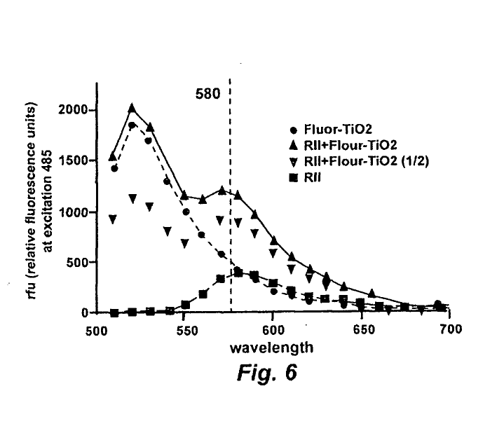

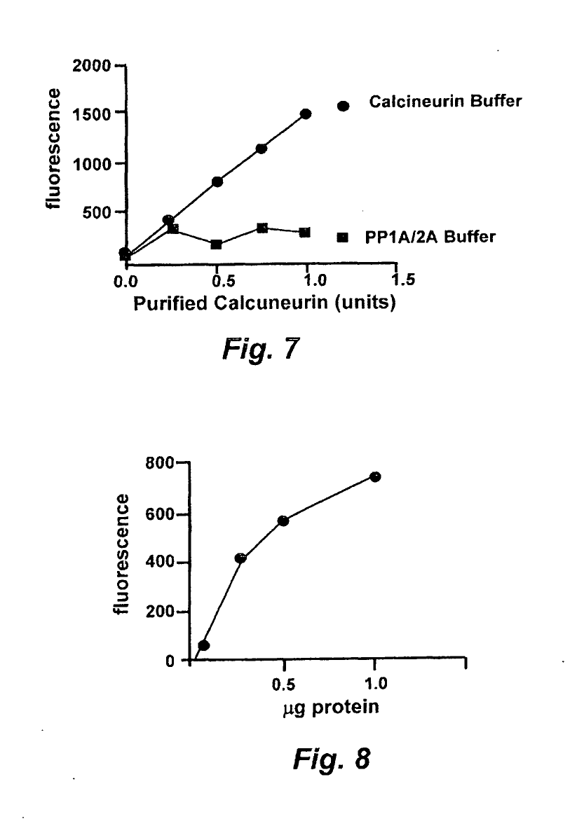

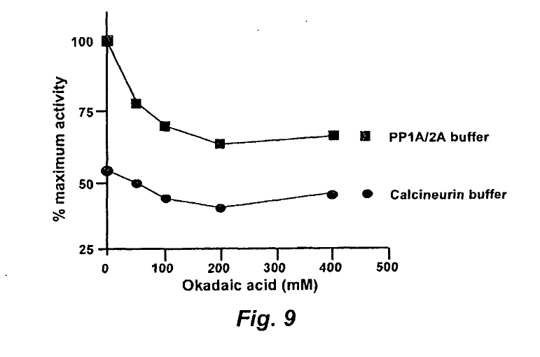

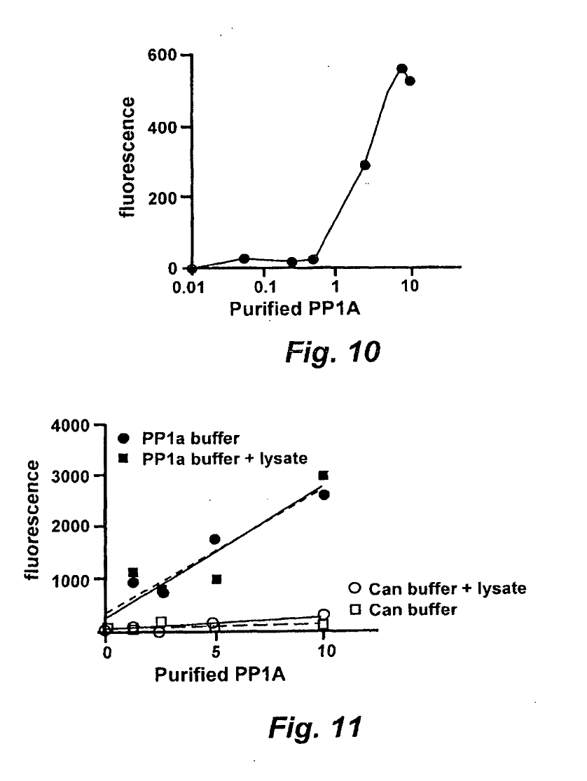

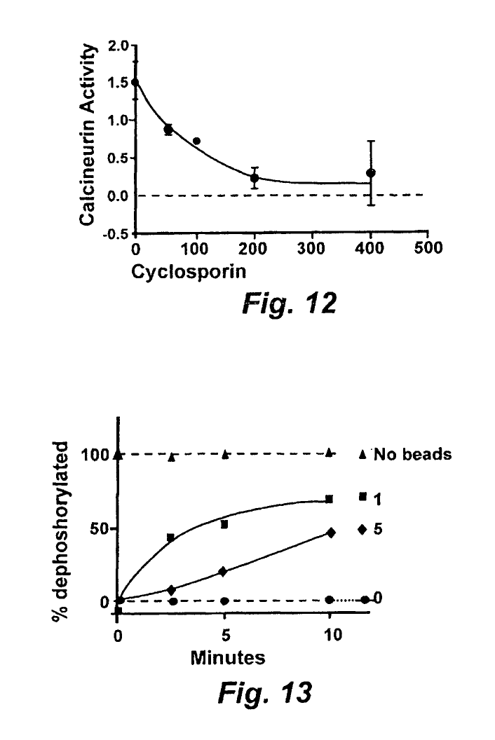

Fluorescent emission from the fluorescein can transfer energy (FRET) to the TAMRA tag, initiating a unique and detectable second fluorescent emission. This transfer only happens when the two cooperating fluorophores are in close physical proximity, i.e. when the phosphorylated peptide is bound to the titanium oxide micro-beads. The second emission peak can then be detected by a fluorimeter and the intensity thereof used to determine the amount of phosphorylated substrate in each reaction. Results of experimental samples can be compared to a standard curve generated with purified calcineurin such as shown in FIG. 5B, or the protein phosphatase PP1 (FIG. 10), and units of enzyme activity calculated accordingly.

Embodiments of the present disclosure, therefore, encompass in-solution FRET-based methods of detecting and quantitatively measuring modifications to proteins or peptides, the modifications introducing or removing acidic moieties conjugated to an amino acid chain. The presence of acidic moieties attached to the polypeptide or peptide allows bonding of the peptide via the acidic group to titanium oxide. Modified peptides, therefore, may be partitioned from unmodified peptides by such as filtration, sedimentation, washing of titanium oxide immobilized to a substratum, and the like.

Unlike the method disclosed in PCT Patent Application Publication No.: WO/2009/010424, incorporated herein by reference in its entirety, the assay according to the present disclosure only detects the secondary fluorescence emitted by FRET, and not of unbound peptide. The light detection apparatus used to detect the fluorescence light may be adapted by the use of such as wavelength-specific filters, to selectively detect one wavelength and not another. Additionally, the reaction using titanium oxide as the method of partitioning modified from unmodified target peptide may be conducted in a single reaction mix and reaction step. The methods of the present disclosure do not require the multi-step procedure of reacting assay components and partitioning bound from unbound peptide before directly determining the amount of the fluorescently labeled peptide not bound to titanium oxide. The method of the present disclosure, therefore, is particularly suitable, and may be readily adapted, for automation.

Also within the scope of the present disclosure is a fluorimeter system for assaying multiple samples for determining the levels therein of a peptide-modifying enzyme having the ability to modify a peptide substrate. Such a system may incorporate a microprocessor, in addition to a fluorimeter, that is operably connected to provide an output of the data that may represent, in a visual form, including such as, but not limited to, a paper printout, an electronically generated image and the like, of the FRET-generated fluorescence intensity value, and its correlation to the activity status of a peptide-modifying enzyme, an inhibitor or activator of such an enzyme and the like.

The present disclosure, therefore, encompasses methods for the determination of enzyme activities that can post-translationally modify a target such as a peptide, an oligopeptide, a polypeptide, or a protein, and particularly of a target that in the modified state may bind to titanium oxide. The methods of the disclosure are especially useful for detecting and measuring the activity of a reaction resulting in the removal of acidic group such as a phospho-group by a phosphatase, or the addition of a phospho-group by a kinase. The assays of the disclosure may further be configured to provide data as to the effect of an effector such as, but not limited to, an inhibitor on the modifying enzyme.

The assay methods of the disclosure provide a target substrate such as, but not limited to, a peptide that has a first fluorophore conjugated thereto. When it is desired to detect or measure the activity of a protein kinase or phosphatase in a sample, for example, a peptide is obtained that has an amino acid sequence specifically recognized by the kinase or phosphatase under the conditions of the assay. To detect a phosphatase activity, the target peptide is further phosphorylated at a site within the peptide that is selectively recognized by the phosphatase when phosphorylated. To determine a kinase activity, a fluorophore-conjugated peptide is provided that comprises a site selectively recognized by the target kinase as a site to be phosphorylated.

In one aspect of the assays of the disclosure, therefore, the target enzyme activity to be detected or determined can be a phosphatase. After allowing the labeled phosphorylated peptide substrate to react with a test sample having a suspected phosphatase activity under conditions allowing phosphatase activity, a mixture of dephosphorylated and phosphorylated peptide will result. The dephosphorylated peptide and the phosphorylated peptide substrate are partitioned by contacting with a titanium oxide matrix that specifically binds the phosphorylated peptide. It is contemplated that the fluorescence measurements may be obtained without the separation of the titanium oxide from the reaction mix since under the FRET-based assay systems of the present disclosure, only phosphate-bound peptides bound to the titanium oxide will be detectable.

Although it is preferred that the micro-beads remain in suspension or at least sediment under gravity, it is contemplated that the micro-beads may be separated from the assay mix supernatant by such as filtration, centrifugation, or by having previously bound the micro-beads to a solid substrate. In the latter case, the reaction mixes maybe contacted to the substrate-bound micro-beads, and after a suitable reaction period the reaction mix may be replaced by a wash solution before determining the FRET fluorescence. The peak of FRET emission can then be detected and used to quantitatively determine the amount of phosphorylated substrate in each reaction. Results of experimental samples are compared to a standard curve generated phosphatase and units of activity calculated.

The amino acid sequence of the peptide substrate may be any sequence that is specific recognized by the peptide-modifying enzyme of interest. It is further contemplated that the sequence may be such as to be capable of distinguishing isoforms of such an enzyme. For example, the substrate peptide may have, but is not limited to, the amino acid sequence according to SEQ ID NOs.: 1 or 2, where the peptides can serve as specific substrates for the phosphatase calcineurin, but SEQ ID NO.: 2 is specific only for one isoform of calcineurin, and not others.

The present disclosure, therefore, in particular provides methods for determining the level of activity of the phosphatase calcineurin in a biological sample derived from a human or animal patient. Embodiments of the assays may be used to determine the response of the calcineurin activity of a patient to a calcineurin inhibitor, which provides predictors for the outcome of transplantation and/or immunosuppression efficacy. Information from the response of the enzyme to a potential inhibitor may further direct the physician to adjust a regimen of therapeutic agents that may increase the acceptance of the patient towards a transplanted organ, and reduce rejection thereof.

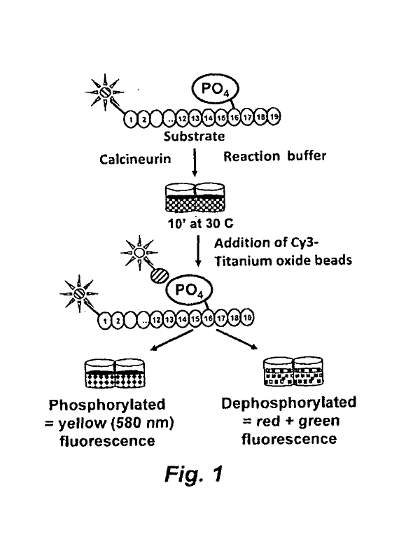

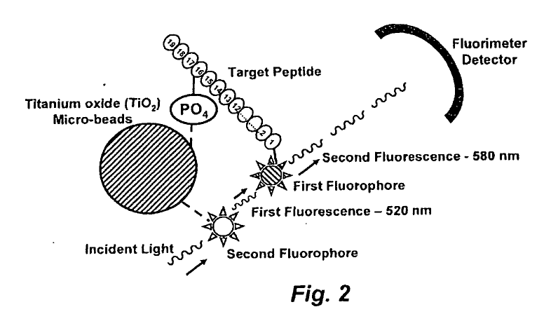

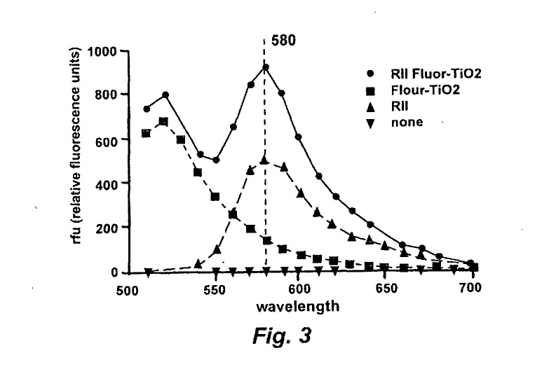

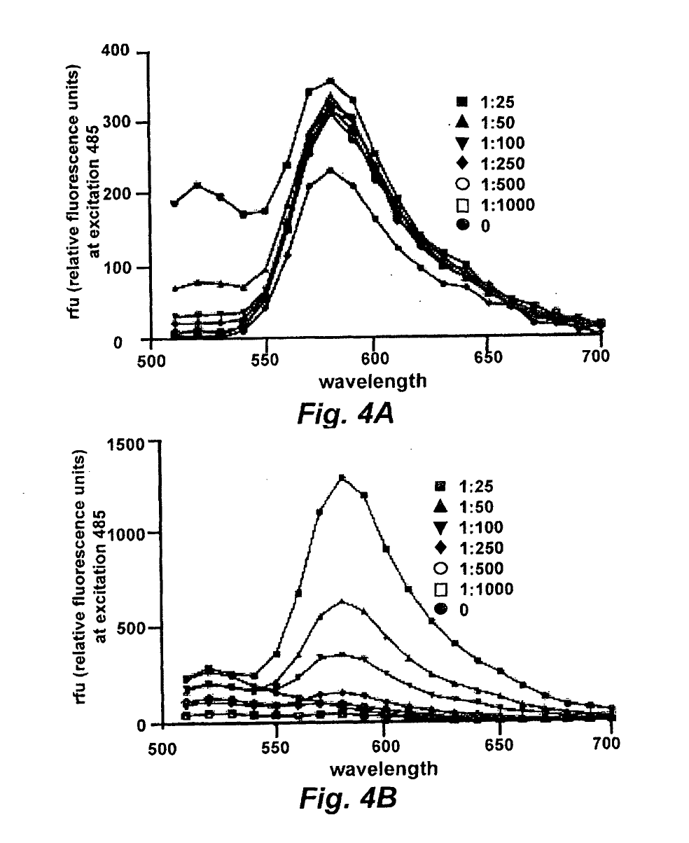

The steps of the methods of the present disclosure are illustrated in FIG. 1. In this example, which illustrates the use of the methods to determine a phosphatase (calcineurin) activity, a peptide is obtained that can be phosphorylated at the Ser-15 position during peptide synthesis itself, thereby eliminating the need for enzymatic labeling. In embodiments of the methods according to the present disclosure, the peptide may have, but is not limited to, the amino acid sequence NH₂-DLDVPIPGRFDRRVSVAAE-COOH (SEQ ID NO.: 1) (the RII peptide), Fluoresceinyl-DLDVPIPGRFDRRVSVAAE, and its phosphorylated analog (Fluoresceinyl-DLDVPIPGRFDRRVpSVAAE where pS=L-phosphoserine) are variants of the peptide SEQ ID NO.: 1 for use in the methods of the disclosure, and in particular for the detection of calcineurin activity. The peptide may also be generated with a fluorescent moiety at its amino-terminus, the fluorescent label being, but not limited to, fluorescein or TAMRA. Next, the labeled target peptide can be incubated with a test sample comprising, for example, a cell lysate, for about 10 mins at about 30° C.

The present disclosure further encompasses assays that use a peptide substrate that can be selectively dephosphorylated by the β isoform of calcineurin and not the α isoform. The amino acid sequence of the isoform-specific peptide substrate is based on a portion of the NFATc protein, a known substrate of calcineurin, which has been modified to improve isoform selectivity and ease of synthesis. The amino acid sequence of the peptide is ASPQTSPWQSPAVSPK (SEQ ID NO.: 2) wherein the Ser-6 position may be phosphorylated. A fluorescently labeled version of the peptide is as follows: ASPQT(pS)PWQSPAVSPK with an N-terminal fluorescent TAMRA group and a C-terminal amide group, although it is contemplated that a fluorescent group other than TAMRA may be substituted without affecting the efficacy of the substrate.

One aspect of the present disclosure encompasses methods for determining a peptide-modifying enzyme activity, the method comprising: (a) providing an assay reaction mix comprising a target peptide comprising an amino acid sequence specifically recognized by a peptide-modifying enzyme and a first fluorophore species conjugated to said target peptide, a buffer mix configured to allow a peptide-modifying enzyme to modify the target peptide, and a test sample suspected of comprising a peptide-modifying enzyme; (b) incubating the assay reaction mix under conditions suitable for a peptide-modifying enzyme to modify the target peptide; (c) contacting the incubated reaction mix with titanium oxide, said titanium oxide having a second fluorophore species conjugated thereon, under conditions suitable for the titanium oxide to bind to a modified target peptide but not to an unmodified target peptide; (d) illuminating the titanium oxide at an excitation wavelength of the second fluorophore species, whereby the second fluorophore species emits a first fluorescence, said first fluorescence exciting the first fluorophore species by FRET, thereby inducing the emission of a second fluorescence from the first fluorophore species, said second fluorescence having a wavelength different from the wavelength of the first fluorescence; (e) selectively detecting the second fluorescence, thereby detecting binding of a modified target peptide to titanium oxide; (f) determining the intensity of the second fluorescence; and (g) correlating the intensity of the second fluorescence to the amount of modified target peptide bound to the titanium oxide.

In some embodiments of this aspect of the disclosure, the methods may further comprise correlating the intensity of the second fluorescence with a level of activity of the peptide-modifying enzyme in the test sample.

In embodiments of this aspect of the disclosure, the target peptide can comprise an acidic modifying group conjugated to the target peptide. In some embodiments, the acidic modifying group can be a phosphate group.

In some embodiments, of the method of the disclosure, the target peptide can be an unmodified peptide, the peptide receiving a modifying group, and the peptide-modifying enzyme attaching the modifying group to the target peptide. In some of these embodiments, the modifying group can be a phosphate group, and the peptide-modifying enzyme is a kinase.

In other embodiments of the methods of the disclosure, the target peptide can have a modifying group attached thereto and the peptide-modifying enzyme can remove the modifying group from the target peptide.

In some of these embodiments, the modifying group can be a phosphate group, and the peptide-modifying enzyme is a phosphatase. In some embodiments of the methods of this aspect of the disclosure, the phosphatase is a calcineurin.

In the embodiments of this aspect of the disclosure, the titanium oxide can be formulated as micro-beads, where the micro-beads are suspended in the assay reaction mix, or immobilized to a substratum.

In certain embodiments of the method, the target peptide can have an amino acid sequence SEQ ID NO.: 1 or SEQ ID NO.: 2.

In the embodiments of the disclosure, the target peptide can selectively distinguishes a first isoform of calcineurin from a second isoform of calcineurin.

In one embodiment of the methods according to the present disclosure, the target peptide may have an amino acid sequence according to SEQ ID NO.: 2, and the target peptide is a phosphorylated variant characterized as being specifically dephosphorylated by the □-isoform of calcineurin.

In one embodiment of the disclosure, the first fluorophore species can be a TAMRA group.

In one embodiment of the disclosure, the second fluorophore species may be an N-terminal fluorescein group.

In yet another embodiment of this aspect of the disclosure, the test sample may be an isolated population of Peripheral Mononuclear Blood Cells (PMBCs), an isolated population of T-cells, or a combination thereof, or a lysate thereof.

In yet other embodiments of the methods of this aspect of the disclosure, the assay methods can further comprise: (i) providing a first test sample and performing steps (a)-(f) on said first test sample, thereby obtaining a first value of the second fluorescence intensity; (ii) providing a second test sample, wherein the second test sample comprises a known amount of an active peptide-modifying enzyme activity, and repeating steps (a)-(f) on said second sample, thereby obtaining a second value of the second fluorescence intensity; and (iii) comparing the first value of the second fluorescence intensity with the second value of the second fluorescence intensity, thereby determining the amount of a peptide-modifying enzyme activity in the first test sample.

In one embodiment, the first test sample can be obtained from a human or animal subject, in need of a transplant or has received a transplant, and the efficacy of a calcineurin inhibitor in the human or animal subject can be determined from the level of calcineurin activity in the first test sample when in the presence of the calcineurin inhibitor. In this embodiment of the method, the method may further comprise: obtaining from a human or animal subject a first test sample and a second test sample; determining the level of activity of calcineurin in the first test sample; determining the level of activity of calcineurin in the second test sample in the presence of a calcineurin inhibitor; and comparing the levels of calcineurin activity in the first and second samples, thereby predicting a response of the human or animal subject to a calcineurin inhibitor administered thereto.

In one embodiment, the prediction of the response of a human or animal subject to an administered calcineurin inhibitor can provide a prognosis of a transplant in the human or animal subject. In this embodiment, the transplant can be a renal transplant.

Another aspect of the present disclosure encompasses kits for determining the level of a peptide-modifying enzyme activity in a test sample, comprising: a container enclosing a target peptide, the target peptide comprising an amino acid sequence specifically recognized by a peptide-modifying enzyme and a first fluorophore species conjugated to the target peptide; titanium oxide conjugated to a second fluorophore species; and instructions for the use of the target peptide and the titanium oxide in determining the peptide-modifying enzyme activity of a test sample by FRET-based fluorescence measurements.

In one embodiment of the kits of this aspect, the titanium oxide can be formulated as micro-beads.

In some embodiments, the target peptide may further comprise an acidic modifying group conjugated to the target peptide.

In some embodiments, the target peptide can be phosphorylated, and the instructions direct the use of the kit to determine a phosphatase activity.

In other embodiments, the target peptide is non-phosphorylated, and the instructions direct the use of the kit to determine a kinase activity.

In one embodiment of this aspect of the disclosure, the target peptide can be capable of being specifically dephosphorylated by the □-isoform of calcineurin and comprises a peptide having the amino acid sequence according to SEQ ID NO.: 2, where the S-6 position is phosphorylated, an N-terminal fluorescent TAMRA group, and a C-terminal amide group.

Yet another aspect of the present disclosure encompasses fluorimetric unit configured to measure a peptide-modifying enzyme activity according to a FRET-based method, comprising: a system configured to receive a plurality of assay reaction mixes, each reaction mix comprising: a target peptide comprising an amino acid sequence specifically recognized by a peptide-modifying enzyme and a first fluorophore species conjugated to said target peptide, a buffer mix configured to allow a peptide-modifying enzyme to modify the target peptide, wherein each assay mix of said plurality of assay mixes is in contact with titanium oxide having a second fluorophore species conjugated thereto, and wherein the titanium oxide is formulated as titanium oxide micro-beads or substratum-attached titanium oxide, a detection system for detecting binding of a modified target peptide to titanium oxide by FRET, wherein the titanium oxide is illuminated at an excitation wavelength of the second fluorophore species, whereby the second fluorophore species emits a first fluorescence, said first fluorescence exciting the first fluorophore species, thereby inducing the emission of a second fluorescence from the first fluorophore species, said second fluorescence having a wavelength different from the wavelength of the first fluorescence, a measuring system configured to determine the intensity of the second fluorescence, a microprocessor unit configured to correlate the intensity of the second fluorescence to the amount of modified target peptide bound to the titanium oxide; and an output unit configured to provide a measurement of the peptide-modifying enzyme activity.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### EXAMPLES

### Example 1

### Materials and Reagents

Recombinant calcineurin was from Calbiochem (San Diego, Calif.), and all other chemicals were obtained from Sigma (St Louis, Mo.). The RII peptide, Fluoresceinyl-DLDVPIPGRFDRRVSVAAE (SEQ ID NO.: 1), and its phosphorylated analog (Fluoresceinyl-DLDVPIPGRFDRRVpSVAAE (SEQ ID NO.: 2) where pS=L-phosphoserine) were synthesized by Fmoc-based solid-phase peptide synthesis using model Liberty microwave-assisted peptide synthesizer (CEM Corporation, Matthews, N.C.). The peptides were purified to apparent homogeneity by reversed-phase HPLC and their masses were confirmed by mass spectrometry. Peptides were diluted in: Tris 50 mM, 100 mM NaCl, 0.5 mM DTT, and 0.1 mg/ml bovine serum albumen to a final concentration of 30 ng/ml. Reaction buffer consisted of: 0.1 mg/ml bovine serum albumen, 35 mM Tris pH 7.5, 25 mM NaCl, 2.0 mM MgCl₂, 270 μM DTT, 500 μM EDTA, 419 nM okadaic acid (in 0.63% ethanol), 25 mM CaCl₂.

### Example 2

### Protocol for Calcineurin in-Solution Assay Method

1. Peripheral mononuclear blood cells (PMBCs) are pelleted, resuspended in a hypotonic lysis buffer (see Gooch et al., J. Biol. Chem. 276 (2001) 42492-500; Gooch et al., (2004) J. Biol. Chem. 279: 15561-70; incorporated herein by reference in their entireties) and then lysed by three rounds of freeze/thawing in liquid nitrogen and a 30° C. water bath. This ensured that the cell membranes were ruptured in an isotonic buffer that did not interfere with calcineurin activity. CD3+/4+ T cells may be further isolated by a FACS-based system

2. Calcineurin activity was assessed by mixing equal parts lysate, RII or other peptide sample), and reacted for 5-10 mins at 30° C. Control reactions were included that contained known varying amounts of purified calcineurin to calculate a standard curve for each plate.

3. To stop the reaction, a solution of 10% acetyl nitrile and 0.01% acetic acid solution was added. This step was essential to reduce the pH of the reaction to below 3.5 and enable the binding of the titanium oxide beads to the target peptide.

4. FLUOR-titanium oxide beads were added with gentle shaking for 5 mins. Phosphorylated-peptides were retained on the beads producing a “shifted” TAMRA signal while non-phosphorylated peptides were unbound.

5. Reactions were neutralized with 0.35% ammonium hydroxide to return the pH to greater than 7 and the fluorescence of each sample will be read at 450 nm excitation/580 nm emission.

6. Calcineurin activity was then determined by calculating the slope and y-intercept of the standard curve and then extrapolating calcineurin activity from the fluorescence intensity.

Hypolonic Lysis Buffer: 50 mM Tris pH 7.5, 1 mM EDTA, 1 mM EGTA, 0.5 mM DTT, 50 μg/ml PMSF, 10 μg/ml leupeptin, 10 μg/ml aprotinin.

Reaction Buffer: 0.1 mg/ml BSA, 35 mM Tris pH 7.5, 25 mM NaCl, 2.0 mM MgCl₂, 270 μM DTT, 500 μM EDTA, 419 nM Okadaic acid (in 0.63% ethanol), 25 mM CaCl₂

1. A method for determining a peptide-modifying enzyme activity, the method comprising:
(a) providing an assay reaction mix comprising:
a target peptide comprising an amino acid sequence specifically recognized by a peptide-modifying enzyme and a first fluorophore species conjugated to said target peptide; a buffer mix configured to allow a peptide-modifying enzyme to modify the target peptide; and
a test sample suspected of comprising a peptide-modifying enzyme;
(b) incubating the assay reaction mix under conditions suitable for a peptide-modifying enzyme to form a modified target peptide;
(c) contacting the incubated reaction mix with titanium oxide, said titanium oxide having a second fluorophore species conjugated thereon, under conditions suitable for the titanium oxide to bind to a modified target peptide but not to an unmodified target peptide;
(d) illuminating the titanium oxide at an excitation wavelength of the second fluorophore species, whereby the second fluorophore species emits a first fluorescence, said first fluorescence exciting the first fluorophore species by FRET, thereby inducing the emission of a second fluorescence from the first fluorophore species, said second fluorescence having a wavelength different from the wavelength of the first fluorescence;
(e) selectively detecting the second fluorescence, thereby detecting binding of a modified target peptide to titanium oxide;
(f) determining the intensity of the second fluorescence; and
(g) correlating the intensity of the second fluorescence to the amount of modified target peptide bound to the titanium oxide.

2. The method of claim 1, further comprising correlating the intensity of the second fluorescence with a level of activity of the peptide-modifying enzyme in the test sample.

3. The method of claim 1, wherein the target peptide is a modified target peptide comprising an acidic modifying group conjugated to the target peptide.

4. The method of claim 3, wherein the acidic modifying group is a phosphate group.

5. The method of claim 1, wherein the target peptide is an unmodified peptide, and wherein said peptide receives a modifying group, the peptide-modifying enzyme attaching the modifying group to the target peptide.

6. The method of claim 1, wherein the target peptide has a modifying group attached thereto, the peptide-modifying enzyme removing the modifying group from the target peptide.

7. The method of claim 5, wherein the modifying group is a phosphate group, and the peptide-modifying enzyme is a kinase.

8. The method of claim 6, wherein the modifying group is a phosphate group, and the peptide-modifying enzyme is a phosphatase.

9. The method of claim 8, wherein the phosphatase is a calcineurin.

10. The method of claim 1, wherein the titanium oxide is formulated as micro-beads, and wherein the micro-beads are suspended in the assay reaction mix.

11. The method of claim 1, wherein the titanium oxide is immobilized on a substratum.

12. The method of claim 1, wherein the target peptide has an amino acid sequence selected from SEQ ID NO.: 1 and SEQ ID NO.: 2.

13. (canceled)

14. The method of claim 9, wherein the target peptide has an amino acid sequence according to SEQ ID NO.: 2, and wherein the target peptide is a phosphorylated variant characterized as being specifically dephosphorylated by the β isoform of calcineurin.

15. The method of claim 1, wherein the first fluorophore species is a TAMRA group.

16. The method of claim 1, wherein the second fluorophore species is N-terminal fluorescein group.

17. The method of claim 1, wherein the test sample is an isolated population of Peripheral Mononuclear Blood Cells (PMBCs), an isolated population of T-cells, or a combination thereof, or a lysate thereof.

18. The method of claim 1, further comprising:
(i) providing a first test sample and performing steps (a)-(f) on said first test sample, thereby obtaining a first value of the second fluorescence intensity;
(ii) providing a second test sample, wherein the second test sample comprises a known amount of an active peptide-modifying enzyme activity, and repeating steps (a)-(f) on said second sample, thereby obtaining a second value of the second fluorescence intensity;
(iii) comparing the first value of the second fluorescence intensity with the second value of the second fluorescence intensity, thereby determining the amount of a peptide-modifying enzyme activity in the first test sample.

19. The method of claim 18, wherein the first test sample is obtained from a human or animal subject, wherein the human or animal subject is in need of a transplant or has received a transplant, and wherein the efficacy of a calcineurin inhibitor in the human or animal subject is determined from the level of calcineurin activity in the first test sample when in the presence of the calcineurin inhibitor.

20-22. (canceled)

23. A kit for determining the level of a peptide-modifying enzyme activity in a test sample, comprising:
a container enclosing a target peptide, the target peptide comprising an amino acid sequence specifically recognized by a peptide-modifying enzyme and a first fluorophore species conjugated to the target peptide;
titanium oxide conjugated to a second fluorophore species; and
instructions for the use of the target peptide and the titanium oxide in determining the peptide-modifying enzyme activity of a test sample by FRET-based fluorescence measurements.

24-28. (canceled)

29. A fluorimetric unit configured to measure a peptide-modifying enzyme activity according to a FRET-based method, comprising:
a system configured to receive at least one assay reaction mix, said at least one reaction mix comprising:
a target peptide comprising an amino acid sequence specifically recognized by a peptide-modifying enzyme and a first fluorophore species conjugated to said target peptide;
a buffer mix configured to allow a peptide-modifying enzyme to modify the target peptide;
wherein each assay mix or a plurality of said assay mixes is in contact with titanium oxide having a second fluorophore species conjugated thereto, and wherein the titanium oxide is formulated as titanium oxide micro-beads or substratum-attached titanium oxide;
a detection system configured for detecting binding of a modified target peptide to titanium oxide by FRET, wherein the titanium oxide is illuminated at an excitation wavelength of the second fluorophore species, whereby the second fluorophore species emits a first fluorescence, said first fluorescence exciting the first fluorophore species, thereby inducing the emission of a second fluorescence from the first fluorophore species, said second fluorescence having a wavelength different from the wavelength of the first fluorescence;
a measuring system configured to determine the intensity of the second fluorescence;
a microprocessor unit configured to correlate the intensity of the second fluorescence to the amount of modified target peptide bound to the titanium oxide; and
an output unit configured to provide a measurement of the peptide-modifying enzyme activity.
